(19) United States
(12) Patent Application Publication
(10) Pub. No.: US 2021/0170195 A1
(43) Pub. Date: Jun. 10, 2021

(54) PHOTOTHERAPY GLASSES AND GLASSES CASE

(71) Applicants: Beijing Xiaomi Mobile Software Co., Ltd., Beijing (CN); Lumos Health Inc., Waterloo (CA)

(72) Inventors: Chengyu Wen, Waterloo (CA); Fei Sun, Beijing (CN)

(73) Assignees: Beijing Xiaomi Mobile Software Co., Ltd.; Lumos Health Inc.

(21) Appl. No.: 16/852,786

(22) Filed: Apr. 20, 2020

(30) Foreign Application Priority Data
Dec. 5, 2019 (CN) 201911235702.7

(51) Int. Cl. A61N 5/06; H02J 7/00; A45C 11/04

(52) U.S. Cl. CPC A61N 5/0622; H02J 7/0047; H02J 50/10; H02J 7/0042; A45C 11/04

(57) ABSTRACT

Phototherapy glasses include: a front frame, a first temple, and a second temple, the first temple and the second temple being hinged to a first side and a second side of the front frame, respectively, so that the first temple and the second temple can be folded and unfolded relative to the front frame; a light source component disposed on at least one of the first temple or the second temple; a lens disposed on the front frame; and a reflecting layer, the reflecting layer being formed on the lens and configured to reflect light from the light source component, so that the light is reflected to eyes of a user.

### CROSS-REFERENCE TO RELATED APPLICATIONS

This application is based upon and claims priority to Chinese Patent Application No. 201911235702.7, filed on Dec. 5, 2019, the entire content of which is incorporated herein by reference.

### TECHNICAL FIELD

The present disclosure generally relates to the technical field of terminals, and more particularly, to phototherapy glasses and a glasses case.

### BACKGROUND

Visible phototherapy can use artificial light to simulate sunlight, to help treating various mental conditions and other symptoms related to the human biological clock, such as seasonal affective disorders, depression, delayed sleep disorders, and insomnia.

In a related art, a phototherapy lamp may be provided. A user needs to sit near the phototherapy lamp and enable light emitted by the phototherapy lamp to reach the user's eyes, so as to achieve the purpose of phototherapy. However, due to the disadvantages of heavy weight of the phototherapy lamp and tedious position movement, the user may not move freely during the therapy.

In another related art, a wearable phototherapy product may be provided. A user can perform some daily activities, such as sweeping a floor and reading a book, while wearing the wearable phototherapy product to achieve the purpose of phototherapy. However, current wearable phototherapy products may have special or strange product forms, which affect the aesthetics and convenience of patients when they are worn.

### SUMMARY

According to a first aspect of embodiments of the disclosure, phototherapy glasses include: a front frame, a first temple, and a second temple, the first temple and the second temple being hinged to a first side and a second side of the front frame, respectively, so that the first temple and the second temple can be folded and unfolded relative to the front frame; a light source component disposed on at least one of the first temple or the second temple; a lens disposed on the front frame; and a reflecting layer, the reflecting layer being formed on the lens and configured to reflect light from the light source component, so that the light is reflected to eyes of a user.

According to a second aspect of the embodiments of the disclosure, a glasses case is provided to cooperate with the phototherapy glasses according to the first aspect. The glasses case includes a charging component. The charging component cooperates with the phototherapy glasses to charge the phototherapy glasses through the glasses case.

It is to be understood that the above general description and detailed description below are only exemplary and explanatory and not intended to limit the present disclosure.

### BRIEF DESCRIPTION OF THE DRAWINGS

The accompanying drawings, which are incorporated in and constitute a part of this specification, illustrate embodiments consistent with the disclosure and, together with the specification, serve to explain the principles of the disclosure.

FIG. 1 is a schematic diagram illustrating phototherapy glasses according to some embodiments of the disclosure.

FIG. 2 is a schematic diagram illustrating wearing of phototherapy glasses according to some embodiments of the disclosure.

FIG. 3 is a schematic diagram illustrating a light source component of phototherapy glasses according to some embodiments of the disclosure.

FIG. 4 is a block diagram illustrating phototherapy glasses according to some embodiments of the disclosure.

### DETAILED DESCRIPTION

Reference will now be made in detail to exemplary embodiments, examples of which are illustrated in the accompanying drawings. The following description refers to the accompanying drawings in which the same reference numeral in different drawings represents the same or similar elements unless otherwise specified. The implementations set forth in the following description of exemplary embodiments do not represent all implementations consistent with the disclosure. Instead, they are merely examples of apparatuses and methods consistent with aspects related to the disclosure as recited in the appended claims.

Terms used in the disclosure are for the purpose of describing exemplary embodiments only, and are not intended to limit the disclosure. For example, although the terms “first,” “second,” etc. may be used in the disclosure to describe various information, such information should not be limited to these terms. These terms are only used to distinguish the same type of information from each other. For example, without departing from the scope of the disclosure, first information may also be referred to as second information and, similarly, second information may also be referred to as first information.

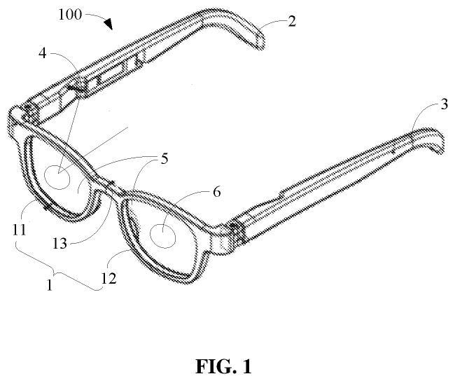

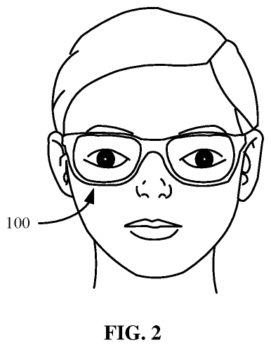

FIG. 1 is a schematic diagram illustrating phototherapy glasses 100 according to some embodiments of the disclosure. As shown in FIG. 1, the phototherapy glasses 100 may include a front frame 1, a first temple 2, and a second temple 3. The first temple 2 and the second temple 3 are hinged to a first side and a second side of the front frame 1, respectively. Therefore, the first temple 2 and the second temple 3 can be folded in a direction close to the front frame 1, and the phototherapy glasses 100 are switched to a folded state and are convenient to carry. Both the first temple 2 and the second temple 3 can be unfolded in a direction away from the front frame 1, and the phototherapy glasses 100 are switched to an unfolded state. Therefore, the phototherapy glasses 100 may be worn on the face of a user as shown in FIG. 2, and phototherapy light may be emitted into the eyes of the user to achieve the phototherapy effect.

Further, the phototherapy glasses 100 may further include a light source component 4, a lens 5, and a reflecting layer 6. The light source component 4 may be disposed on at least one of the first temple 2 or the second temple 3. The lens 5 may be disposed on the front frame 1. The reflecting layer 6 may be formed on the lens 5. The reflecting layer 6 may be configured to reflect light from the light source component 4, so that the light from the light source component 4 can be reflected to the eyes of the user to achieve the phototherapy effect. The phototherapy glasses 100 may further include a color changing layer formed on the lens 5. The color changing layer may change color according to the intensity of irradiated ultraviolet light. For example, when the ultraviolet light is strong, the color of the color changing layer is darkened, and light entering the eyes of the user is weakened. When the ultraviolet light is weak, the color of the color changing layer is lighter, which is beneficial to light transmission and makes the eyes of the user more adaptable to current environmental conditions.

The technical solutions provided by the embodiments of the disclosure may include the following beneficial effects.

In the above embodiments, the phototherapy glasses 100 can be stored and worn through the folding and unfolding of the first temple 2 and the second temple 3 relative to the front frame 1. The structure is simplified as compared to the phototherapy glasses in the related art. Further, the light source component 4 is disposed on the first temple 2 or the second temple 3 and cooperates with the reflection effect of the reflecting layer 6 to simplify the appearance structure of the phototherapy glasses while achieving the phototherapy effect.

In some embodiments, the reflecting layer 6 may be disposed on a side of the lens 5 facing the eyes of the user, thereby avoiding the light from the light source component 4 from being weakened or refracted during the process of light transmission through the lens, which is beneficial to ensure the phototherapy effect. Further, the reflecting layer 6 may allow light of a fixed frequency to be reflected, and light of a frequency other than the fixed frequency may pass through the reflecting layer 6, so that the user can see the outside world with the phototherapy glasses while ensuring the phototherapy effect.

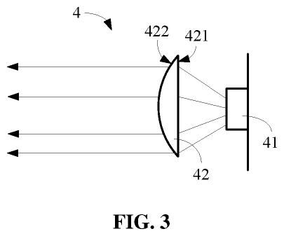

In an embodiment, as shown in FIG. 3, the light source component 4 may include a light source 41 and a plano-convex lens 42. The plano-convex lens 42 includes a light incident surface 421 and a light exit surface 422. Light emitted by the light source 41 may enter the light incident surface 421 of the plano-convex lens 42 and may exit from the light exit surface 422 perpendicularly. Based on this, the scattered light emitted by the light source 41 may be converted to mutually parallel light, and further into linear light that can be emitted to the reflecting layer 6, thereby reducing the amount of light loss.

In this embodiment, the light source 41 may be disposed at a focal point of the lens 5, and an angle formed by parallel light exiting from the plano-convex lens 42 and the lens 5 may be between 25° and 55°. Therefore, the light from the light source 41 may be emitted to the reflecting layer 6 obliquely. By adjusting the position of the reflecting layer 6 and an incident angle of the light, it is beneficial to make the light from the light source enter the eyes of the user perpendicularly, thereby improving the visual effect and the phototherapy effect. The light source 41 may include an LED light source, such as blue LED light of 465 nm, blue light with the illumination intensity of 1001 ux, green light with the illumination intensity of 3501 ux or white light with the illumination intensity of 1000-50001 ux, which may be specifically designed according to therapy requirements and will not be limited in the disclosure.

In some embodiments, the reflecting layer 6 may include a non-metallic dielectric film layer, which is beneficial to realize the connection between the reflecting layer 6 and the lens 5. For example, the reflecting layer 6 may include a dichroic coating or a dielectric coating. When the reflecting layer 6 is a dielectric coating, for example, a film layer may include 25-30 layers, and the thickness of the reflecting layer 6 is, for example, 2 um±1 um. The reflecting layer may be specifically designed as required and will not be limited in the disclosure.

In some embodiments of the disclosure, as shown in FIG. 1, the front frame 1 may include a first support frame 11, a second support frame 12, and a connecting member 13 connecting the first support frame 11 and the second support frame 12. The first support frame 11 may be hinged to the first temple 2, and the second support frame 12 may be hinged to the second temple 3. Moreover, the first support frame 11 and the second support frame 12 may include openings, respectively, so that the lens 5 may be fitted into the openings and fixed. For example, the lens 5 may include a first lens disposed in the first support frame 11 and a second lens disposed in the second support frame 12.

In some embodiments, in order to equalize the light intensity felt by the two eyes of the user, the first lens and the second lens may each be provided with the reflecting layer 6, and the first temple 2 and the second temple 3 may each be provided with the light source component 4. The switching states of the light source components 4 disposed on the first temple 2 and the second temple 3 may be synchronized to equalize light reflected into different eyes of the user.

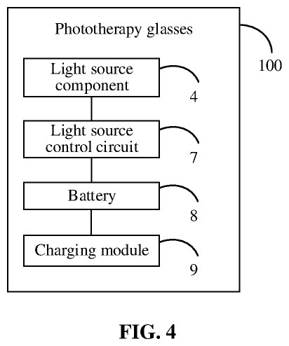

In some embodiments, the light source 41 may include a first light source located on the first temple 2 and a second light source located on the second temple 3. Further, as shown in FIG. 4, the phototherapy glasses 100 may further include a light source control circuit 7 and a battery 8. In one embodiment, the battery 8 may be located in the first temple 2 or the second temple 3, a first end of the light source control circuit 7 may be electrically connected to the battery 8, and a second end of the light source control circuit 7 may be connected to the first light source and the second light source, so that the first light source and the second light source are powered by the battery 8. In another embodiment, the battery 8 may include a first battery in the first temple 2 and a second battery in the second temple 3. Similarly, the light source control circuit 7 may include a first circuit located in the first temple 2 and a second circuit located in the second temple 3. The first circuit is connected to the first light source and the first battery, and the second circuit is connected to the second light source and the second battery, so that the first battery supplies power to the first light source, and the second battery supplies power to the second light source.

Further, as shown in FIG. 4, the phototherapy glasses 100 may further include a charging module 9. The charging module 9 is electrically connected to the battery 8, so that the battery 8 can be charged when the charging module 9 is connected to an external power source. The external power source may be an external power socket or a power bank or another discharge terminal.

In order to protect the phototherapy glasses 100 during non-therapy periods, the disclosure also provides a glasses case (not shown in the figure). The glasses case may cooperate with the phototherapy glasses 100 in each of the above embodiments. Furthermore, the glasses case may further include a charging component. The charging component may cooperate with the phototherapy glasses 100, so that the phototherapy glasses 100 can be charged through the glasses case, and a situation of insufficient power when a user starts the phototherapy glasses 100 can be avoided. In an embodiment, the charging component may include a physical charging interface. The physical charging interface is electrically connected to the charging module 9 of the phototherapy glasses 100 for charging. The physical charging interface may include a USB-C type interface. In another embodiment, the charging component may also charge the phototherapy glasses by wireless charging. For example, the charging component includes an electromagnetic induction type wireless charging coil and forms a charging area inside the glasses case. When the phototherapy glasses 100 are placed in the glasses case and the charging module 9 of the phototherapy glasses 100 is aligned with the charging area in the glasses case, charging can be realized, and the use of a data cable is avoided, which is convenient and quick. Alternatively, the phototherapy glasses may also be wirelessly charged by being attached to the glasses case in a contact manner.

In one embodiment, the glasses case itself may also be charged in a wired or wireless manner, which is not limited here.

In one embodiment, the glasses case may further include an indicator light for displaying a charging state of the phototherapy glasses. The glasses case may also include a display screen for displaying the current power of the phototherapy glasses.

Other embodiments of the disclosure will be apparent to those skilled in the art from consideration of the specification and practice of the disclosure disclosed here. The disclosure is intended to cover any variations, uses, or adaptations of the disclosure following the general principles thereof and including common knowledge or conventional technical means in the art not disclosed in the disclosure. It is intended that the specification and examples be considered as exemplary only, with a true scope and spirit of the disclosure being indicated by the following claims.

It will be appreciated that the disclosure is not limited to the exact construction that has been described above and illustrated in the accompanying drawings, and that various modifications and changes can be made without departing from the scope thereof. It is intended that the scope of the disclosure is only limited by the appended claims.

What is claimed is:

1. Phototherapy glasses, comprising:
a front frame, a first temple, and a second temple, the first temple and the second temple being hinged to a first side and a second side of the front frame, respectively, so that the first temple and the second temple are able be folded and unfolded relative to the front frame;
a light source component disposed on at least one of the first temple or the second temple;
a lens disposed on the front frame; and
a reflecting layer, the reflecting layer being formed on the lens and configured to reflect light from the light source component, so that the light is reflected to eyes of a user.

2. The phototherapy glasses according to claim 1, wherein the reflecting layer is disposed on a side of the lens facing the eyes of the user.

3. The phototherapy glasses according to claim 1, wherein the light source component comprises a light source and a plano-convex lens, the plano-convex lens comprises a light incident surface and a light exit surface, and light emitted by the light source enters the light incident surface of the plano-convex lens and exits from the light exit surface perpendicularly.

4. The phototherapy glasses according to claim 3, wherein the light source is located at a focal point of the lens.

5. The phototherapy glasses according to claim 3, wherein an angle formed by the light exiting from the light exit surface and the lens is between 25° and 55°.

6. The phototherapy glasses according to claim 1, wherein the reflecting layer is a non-metallic dielectric film layer.

7. The phototherapy glasses according to claim 6, wherein the reflecting layer comprises at least one of a dichroic coating or a dielectric coating.

8. The phototherapy glasses according to claim 1, wherein the front frame comprises a first support frame, a second support frame, and a connecting member connecting the first support frame and the second support frame, the first support frame being hinged to the first temple, and the second support frame being hinged to the second temple;
the lens comprises a first lens disposed on the first support frame and a second lens disposed on the second support frame; and
the first temple and the second temple are each provided with the light source component, and the first lens and the second lens are each provided with the reflecting layer.

9. The phototherapy glasses according to claim 1, wherein the light source component comprises a first light source located in the first temple and a second light source located in the second temple; and
the phototherapy glasses further comprise a light source control circuit and a battery, the battery supplying power to the first light source and the second light source through the light source control circuit.

10. The phototherapy glasses according to claim 9, further comprising a charging module electrically connected to the battery and configured to charge the battery.

11. The phototherapy glasses according to claim 1, wherein the phototherapy glasses further comprise a color changing layer formed on the lens.

12. The phototherapy glasses according to claim 3, wherein the light source comprises an LED light source.

13. The phototherapy glasses according to claim 8, wherein the first support frame and the second support frame comprise openings, respectively, so that the first lens and the second lens are fitted into the openings and fixed.

14. The phototherapy glasses according to claim 8, wherein switching states of light source components disposed on the first temple and the second temple are synchronized to equalize light reflected into the eyes of the user.

15. The phototherapy glasses according to claim 9, wherein the battery is located in at least one of the first temple or the second temple, a first end of the light source control circuit is electrically connected to the battery, and a second end of the light source control circuit is connected to the first light source and the second light source.

16. The phototherapy glasses according to claim 9, wherein the battery comprises a first battery in the first temple and a second battery in the second temple, and the light source control circuit comprises a first circuit located in the first temple and a second circuit located in the second temple.

17. The phototherapy glasses according to claim 16, wherein the first circuit is connected to the first light source and the first battery, and the second circuit is connected to the second light source and the second battery, so that the first battery supplies power to the first light source, and the second battery supplies power to the second light source.

18. A glasses case, cooperating with phototherapy glasses, the phototherapy glasses comprising: a front frame, a first temple, and a second temple, the first temple and the second temple being hinged to a first side and a second side of the front frame, respectively, so that the first temple and the second temple are able be folded and unfolded relative to the front frame; a light source component disposed on at least one of the first temple or the second temple; a lens disposed on the front frame; and a reflecting layer, the reflecting layer being formed on the lens and configured to reflect light from the light source component, so that the light is reflected to eyes of a user, and the glasses case comprising:
a charging component, the charging component cooperating with the phototherapy glasses to charge the phototherapy glasses through the glasses case.

19. The glasses case according to claim 18, wherein the charging component comprises at least one of a charging interface or a wireless charging coil.

20. The glasses case according to claim 18, wherein the glasses case further comprises an indicator light for displaying a charging state of the phototherapy glasses.
